(19) United States
(12) Patent Application Publication
(10) Pub. No.: US 2024/0099884 A1
(43) Pub. Date: Mar. 28, 2024

(54) PHACOEMULSIFICATION SYSTEM AND METHOD

(71) Applicant: JOHNSON & JOHNSON SURGICAL VISION, INC., Irvine, CA (US)

(72) Inventors: Assaf Govari, Haifa (IL); Vadim Gliner, Haifa (IL)

(21) Appl. No.: 17/954,613

(22) Filed: Sep. 28, 2022

(51) Int. Cl. A61F 9/007; A61M 1/00

(52) U.S. Cl. CPC A61F 9/00745; A61M 1/73; A61M 1/772; A61B 2217/005; A61B 2217/007; A61M 2205/3344; A61M 2210/0612

(57) ABSTRACT

A phacoemulsification system, method and computer program product, the system comprising: a phacoemulsification probe having a needle at its distal end, the needle configured to be inserted into an eye of a patient, the probe comprising an ultrasonic transducer; a signal generator, meter, and impedance determination module for determining an impedance between a tip of the needle and an electrode attached to the patient; and a processor, configured to repeatedly: obtain the impedance from the impedance detector; subject to the impedance complying with a condition and the ultrasonic transducer being inactive, toggling the ultrasonic transducer to transmit ultrasonic waves; and subject to the impedance not complying with a condition and the ultrasonic transducer being active, toggling the ultrasonic transducer to stop transmitting ultrasonic waves.

### FIELD OF THE DISCLOSURE

This disclosure relates generally to cataract removal, and specifically to a method for operating an ultrasonic transducer during a phacoemulsification operation.

### BACKGROUND OF THE DISCLOSURE

A cataract is a clouding and hardening of the eye's natural lens, which often happens when people get older. A common treatment of cataract is phacoemulsification cataract surgery. In the procedure, a portion of the anterior surface of the lens capsule is removed to gain access to the cataract. The surgeon then uses a phacoemulsification probe, which has an ultrasonic handpiece with a needle. The tip of the needle vibrates at ultrasonic frequency, which emulsifies the cataract lens. At a same time, a pump aspirates particles and fluid from the eye through the tip, wherein the aspirated fluids are replaced with irrigation of a balanced salt solution to maintain the intraocular pressure in the anterior chamber of the eye. After removing the cataract with phacoemulsification, the softer outer lens cortex is removed with suction. An intraocular lens (IOL) is then introduced into the empty lens capsule restoring the patient's vision.

### BRIEF DESCRIPTION OF THE DRAWINGS

The present disclosure will be more fully understood from the following detailed description of the examples thereof, taken together with the drawings, in which:

FIG. 1 is a schematic, pictorial view, along with an orthographic side view, of a phacoemulsification apparatus, in accordance with an example of the present disclosure;

FIG. 2A is a schematic block diagram of a system for activating the ultrasound transducer of a phacoemulsification system according to an impedance measurement, in accordance with some exemplary embodiments of the disclosure;

FIG. 2B and FIG. 2C show two exemplary positions of a needle within an eye of a patient and the associated impedance, in accordance with some exemplary embodiments of the disclosure; and

FIG. 3 is a flowchart of steps in a method for operating an ultrasonic transducer during a phacoemulsification operation, in accordance with some exemplary embodiments of the disclosure.

### DETAILED DESCRIPTION OF EXAMPLES

In the following description, numerous specific details are set forth in order to provide a thorough understanding of the present invention. It will be apparent to one skilled in the art, however, that the present invention may be practiced without these specific details. In other instances, well-known circuits, control logic, and the details of computer program instructions for conventional algorithms and processes have not been shown in detail in order not to obscure the present invention unnecessarily.

Software programming code, which embodies aspects of the present invention, is typically maintained in permanent storage, such as a computer readable medium. In a client-server environment, such software programming code may be stored on a client or a server. The software programming code may be embodied on any of a variety of known media for use with a data processing system. This includes, but is not limited to, magnetic and optical storage devices such as disk drives, magnetic tape, compact discs (CD's), digital video discs (DVD's), and computer instruction signals embodied in a transmission medium with or without a carrier wave upon which the signals are modulated. For example, the transmission medium may include a communications network, such as the Internet. In addition, while the invention may be embodied in computer software, the functions necessary to implement the invention may alternatively be embodied in part or in whole using hardware components such as application-specific integrated circuits or other hardware, or some combination of hardware components and software.

In the description below, the term “about” as related to numerical values may include values that are in the range of +/−10% of the indicated value.

### Overview

Cataract is often handled by a Phacoemulsification (hereinafter “phaco”) procedure, in which an ultrasonic handpiece is used to insert a needle into the patient's eye, wherein the tip of the needle can vibrate at ultrasonic frequency, to emulsify the cataract.

However, operating the ultrasound transducer over the entire procedure is energy consuming, wasteful, and may have negative repercussions on the patient, without any advantage.

Optionally, the ultrasound transducer is manually activated and deactivated by the operating physician, using for example a foot pedal, a switch, or the like. The physician may activate and deactivate the ultrasound transducer based on visual inspection of the needle location relative to the lens, i.e., the physician activates the ultrasound transducer when the needle is in contact with the lens, and deactivates it when the needle is not in contact with the lens.

Since the eye area is rather small, identifying the exact timing at which the needle makes contact with the lens may be difficult to ascertain and may lead to activating the ultrasound transducer before or after contact is made, or deactivating the ultrasound transducer before or after contact is lost. Each of these cases may result in suboptimal results, for example the operation may be less efficient and may take longer.

Therefore, it may be beneficial to automate toggling of the ultrasonic transducer on and off when the needle makes or loses, respectively, contact with the lens being emulsified, such that the operation will be performed in a more efficient manner and without repercussions on the patient.

In order to limit the operation of the ultrasonic transducer to the contact times, it is required to detect when the needle is in contact with the lens. The ultrasonic transducer can then be operated only at the required periods of time, when it can promote the effect of emulsifying the lens.

During the procedure, contact between the needle and the lens is typically intermittent, i.e., the needle is alternately in and out of contact with the lens. Therefore, identifying the engagement and the disengagement of the needle from the lens needs to be performed with a fast response time, i.e., with minimal latency, such that the ultrasonic transducer will be active substantially in synchronization with the needle being in contact with the lens. This is particularly helpful in utilizing even short periods of time during which the needle is in contact with the lens to effect emulsification, and avoiding ineffective activation when there is no contact.

In some exemplary embodiments, the contact between the needle and the lens may be assessed by measuring the impedance between the needle and an electrode positioned on the patient's body. In general, bodily fluids such as the fluid surrounding the lens may have lower electrical resistance than body tissue. Thus, the impedance between the needle and the electrode may be higher as the needle engages with the tissue, in this case the lens, than when the needle is not in contact with the lens. For example, when the needle is in contact with the lens, the impedance between the needle and the electrode may be about 90 Ohm to 110 Ohm, e.g., 100 Ohm, as compared to about 60 Ohm-80 Ohm, e.g., 70 Ohm when the needle is not in contact with the lens.

Whether the impedance indicates that the needle is contacting the lens or not, may be determined in a multiplicity of ways. Generally, such determination may be based upon the existence of one or more conditions.

In one example, an impedance threshold may be determined, for example about 75 Ohm-90 Ohm, e.g., 85 Ohm. If the impedance is above the threshold, it is assumed that the needle is in contact with the lens, and the ultrasonic transducer should be active. The ultrasound transducer may keep operating as long as the impedance is above the threshold. When the impedance drops below the threshold, it is assumed that the needle disengaged from the lens, and the ultrasound transducer may be deactivated. The ultrasound transducer may then be restarted once the needle resumes contact with the lens and the impedance increases again to a value exceeding the threshold.

In another example, since the initial state of the needle is that it is not in contact with the lens, then a baseline of the impedance upon the needle entering the eye is expected to be low. When the impedance increases above the baseline in at least a predetermined ratio, for example about 8%-25%, e.g., 20%, it may be determined that the needle has made contact with the lens, and vice versa.

In yet another example, when the impedance increases in at least a predetermined extent above the baseline, for example about 5 Ohm-30 Ohm, e.g., 15 Ohm relative to the initial impedance, it may be determined that the needle has made contact with the lens, and vice versa.

In further embodiments, the rate of change in impedance may also be considered, such that a sharp change, e.g., the impedance derivative exceeds a first predetermined value or falls below a second predetermined value, it may be determined that the needle engaged with or disengaged from the lens, respectively.

In some embodiments, determining whether the needle is in contact with the lens may be based on an artificial intelligence (AI) engine, wherein the engine is trained on a plurality of measurements performed over a plurality of operations. For example, supervised learning may be employed, in which during training the impedance is measured while a phacoemulsification operation is taking place, and whether the physician is activating the ultrasound transducer or not is construed as a label (also referred to as ground truth) of the needle being in contact with the lens or not, respectively. Then, when the AI engine is used, it may receive as input the impedance, and output a probability that the needle is in contact with the lens.

Determining whether the needle is in contact with the lens or not, and toggling ON/OFF the ultrasound transducer may be performed by a controller, such as a dedicated microcontroller, rather than a central processing unit (CPU) of the apparatus, so as to enable fast response time.

In some embodiments, a drive module may operate and control the ultrasound transducer, wherein the controller and the drive module may be implemented as a single controller or as separate controllers.

The solution provides for activating the ultrasound transducer only when its operation is effective in emulsifying the cataract, thereby saving energy, as well as avoiding negative repercussions on the patient.

Additionally, the rate of change of the impedance value when the needle makes or loses contact with the lens is high relative to the rate of motion of the physician holding the device. Therefore, the fast response time enabled by determining the impedance, provides for using the system in an efficient manner, utilizing even short periods of time in which the needle is in contact with the lens.

### System Description

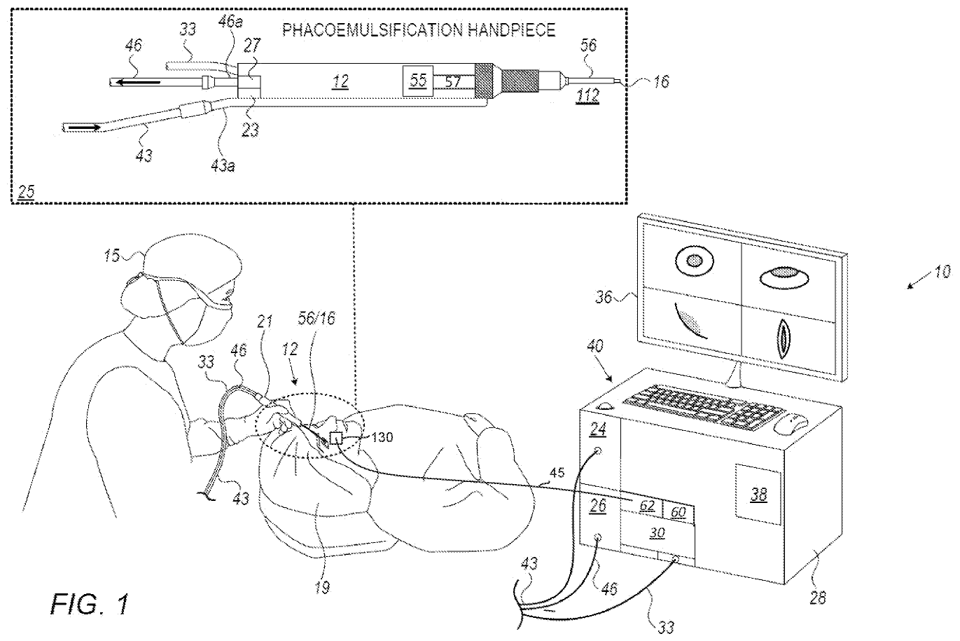

Referring now to FIG. 1, showing an exemplary phacoemulsification apparatus 10, which includes a phacoemulsification probe 12 having a needle 16 at its distal end 112, wherein needle 16 is configured to be inserted by a physician 15 into the lens capsule of an eye of a patient 19 to remove a cataract lens. While needle 16 is shown in inset 25 as a straight object, it is appreciated that any suitable needle may be used with phacoemulsification probe 12, for example, a curved or bent tip needle commercially available from Johnson & Johnson Surgical Vision, Inc., Irvine, CA, USA.

System 10 may comprise console 28, comprising a user interface 40, including physical and virtual controls such as a keyboard, a mouse, a touchscreen, a joystick, a foot pedal, a speaker, a microphone, or others, for inputting data or commands to the apparatus, and a processor 38.

Probe 12 may comprise ultrasound transducer 55, e.g., a piezoelectric ultrasound transducer, which is configured to vibrate horn 57 and needle 16 in one or more resonant vibration modes of the combined horn and needle element. During the phacoemulsification procedure, upon the application of one or more drive signals to ultrasound transducer 55, the vibration of needle 16 is used to emulsify the cataract. Ultrasound transducer 55 and horn 57, or different combinations providing the same effect are collectively referred to as an ultrasound transducer.

In some embodiments, probe 12 may further comprise a coaxial irrigation sleeve 56 that at least partially surrounds needle 16. During the phacoemulsification procedure, an irrigation pump 24 which may be controlled by processor 38 detailed below may pump irrigation fluid from an irrigation reservoir (not shown) to irrigation sleeve 56, to irrigate the eye. The fluid may be pumped via an irrigation tubing line 43 running from console 28 to an irrigation channel 43a of probe 12.

In some embodiments, eye fluid and waste matter (e.g., emulsified parts of the cataract) are aspirated via a hollow in needle 16 to a collection receptacle (not shown) by an aspiration pump 26, also controlled by processor 38, using aspiration tubing line 46 running from aspiration channel 46a of probe 12 to console 28.

Irrigation pump 24 may be controlled by processor 38 in accordance with readings received from irrigation sensor 23, and aspiration pump 26 may be controlled by processor 38 in accordance with readings received from aspiration sensor 27, to maintain intraocular pressure (IOP) within predetermined limits.

Processor 38 is thus adapted to control the operation of various functions of probe 12 such as the irrigation and aspiration, in accordance with the physician's commands as provided via user interface 40, and with various measurements.

It is appreciated that some or all of the functions of processor 38 may be combined in a single physical component. Alternatively, processor 38 may be implemented using multiple physical components. These physical components may comprise hard-wired or programmable devices, or a combination thereof. In some examples, at least some of the functions of processor 38 may be carried out by suitable software stored in a memory device of processor 38 or console 28. This software may be downloaded to a device in electronic form, over a network, or the like. Alternatively, or additionally, the software may be stored in tangible, non-transitory computer-readable storage media, such as optical, magnetic, or electronic memory.

In an example, the apparatus may comprise a display 36 for displaying various images and aspects of the operation to the physician. In some embodiments, one or more controls of user interface 40 and display 36 may be integrated into a touch screen graphical user interface.

The system may comprise drive module 30 for activating ultrasound transducer 55, for example setting the operation parameters and supplying current to ultrasound transducer 55. Drive module 30 may be realized in hardware or software, for example, in a proportional-integral-derivative (PID) control architecture.

The system may comprise impedance detection circuitry 62, for monitoring the impedance between needle 16 and electrode 130 such as a patch electrode, attached to patient 19. Electrode 130 may be relatively small, for example a patch of about 1 cm×1 cm, and attached to patient 19 in a location close to eye 20, such as on or near the patient's nose, forehead, cheek, or the like. However, electrode 130 may be attached to patient 19 in any other area on the body. Electrode 130 may be made of any conductive material, such as Copper, Platinum Iridium, or the like.

The system may comprise controller 60 configured to determine based on the impedance obtained by impedance detection circuitry 62 whether needle 16 is in contact with the lens. According to whether contact is made or lost, controller 60 may toggle ultrasound transducer 55 ON or OFF.

Additionally, in some embodiments, physician 15 may use any control of user interface 40 to set a vibration mode and/or frequency of ultrasound transducer 55, or to override the automatic activation and deactivation by manually starting or stopping the ultrasonic transducer.

The apparatus shown in FIG. 1 may include further elements, which are omitted for clarity of presentation. For example, physician 15 typically performs the procedure using a stereo microscope or magnifying glasses, neither of which are shown. Physician 15 may use other surgical tools in addition to probe 12, which are also not shown in order to maintain clarity and simplicity of presentation.

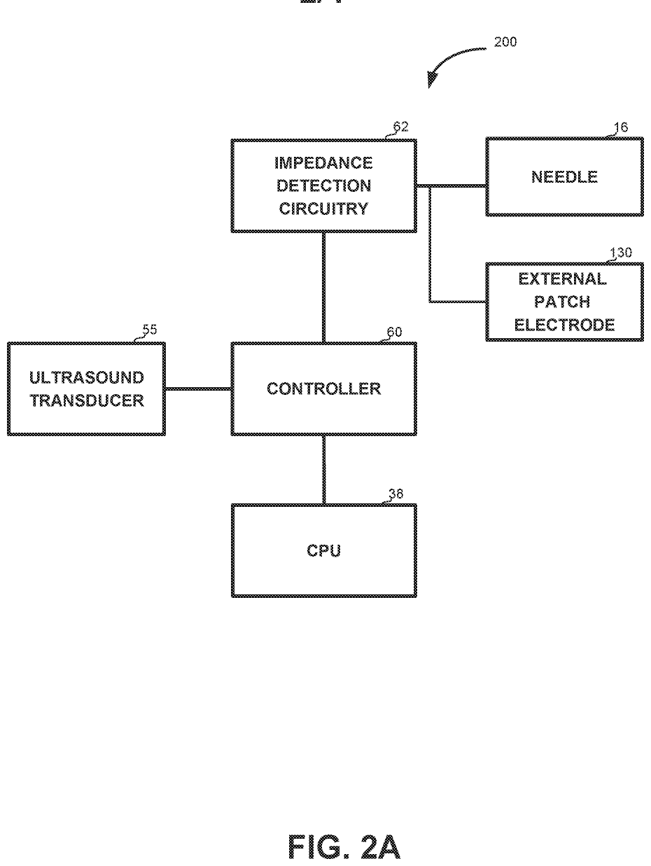

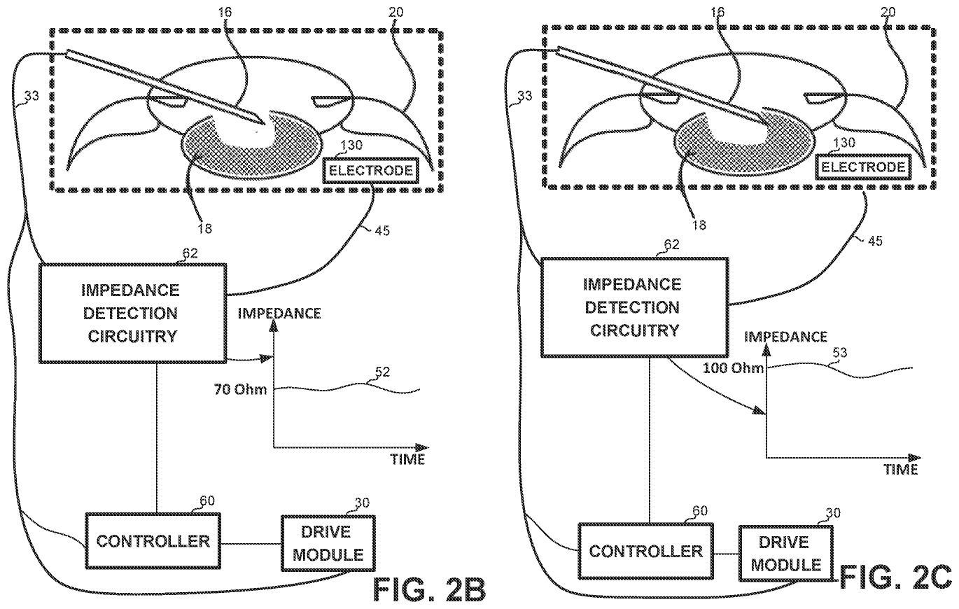

Referring now to FIG. 2A, showing a schematic block diagram of an ultrasound activation system 200 for activating an ultrasound transducer of a phacoemulsification system according to the impedance, in accordance with some exemplary embodiments of the disclosure.

System 200 may comprise impedance detection circuitry 62, for generating a defined signal between needle 16 or an electrode attached thereto, and electrode 130, and monitoring the impedance based on methods known in the art, e.g., measuring current or power and determining the ratio between the voltage and the current.

Impedance detection circuitry 62 may comprise a signal generator for applying a small-amplitude signal to needle 16 and to electrode 130. The signal may be, for example, between 5 μAmp and 100 μamp, such as 20 μamp, and at a frequency between 10 KHz and 100 KHz, for example 50 KHz. The signal may be non-excitory, i.e., a signal that does not excite the body tissue and does not cause muscle contraction.

Impedance detection circuitry 62 may comprise a meter for monitoring the voltage generated between probe 12 and electrode 130 in response to the signal transmitted by the signal generator. Since the frequency of the input is known, it may be used for monitoring the voltage.

Impedance detection circuitry 62 may determine the impedance between needle 16 and electrode 130, for example by dividing the voltage by the current. In some embodiments, the signal generator may apply multiple signals at multiple frequencies, or a signal of varying frequency, and the meter may take a measure for each such frequency. The overall impedance may then be computed by considering the multiple impedances measured for the multiple frequencies.

Impedance detection circuitry 62 may be adapted to detect impedance in the range of 20-150 Ohm, 50-120 Ohm, 20-120 Ohm, 50-150 Ohm, or the like.

In some embodiments, signal generator 64 may apply voltage, for example in the range of 0.1V to 3V, such as 0.5V and meter 65 may measure the current through the circuit, for example using a current sensing resistor, to obtain the impedance.

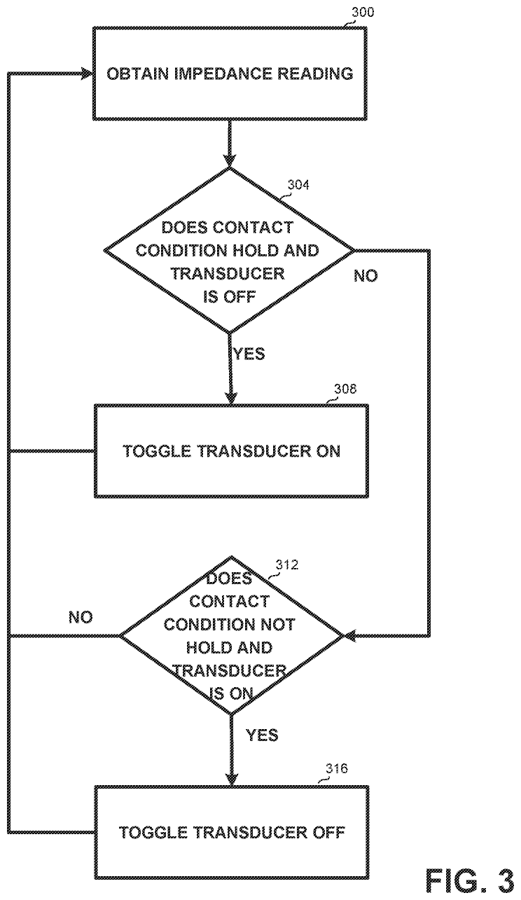

The impedance may change according to whether probe 12 touches or loses touch with the lens, thus by monitoring the impedance controller 60 may determine whether probe 12 is in contact with the lens or not, by testing one or more conditions as described in association with FIG. 3 below.

Controller 60 may be configured to receive input from impedance detection circuitry 62 and may determine whether the impedance indicates that the needle is in contact with the eye or not, and toggle ultrasound transducer 55 ON/OFF accordingly.

Controller 60 may be a microcontroller such as a dedicated microcontroller. Controller 60 may thus be a 32-bit microcontroller with fast, e.g., sub-microsecond real-time capabilities, digital signal processing, low-power/low voltage operation, and connectivity, while maintaining a small form factor for example, the STM32 microcontroller, made by STM, which includes a DMA memory and can be used to control powering of the ultrasound transducer. Controller 60 may have processing and memory capabilities and may be configured for toggling ultrasound transducer 55 without the need to engage processor 38 and its associated firmware/software, and thus reduce delays and provide faster response time. However, in some embodiments, controller 60 may also receive commands from processor 38 of console 28.

Controller 60 may be integrated into console 28. In yet further examples, controller 60 may be integrated into phacoemulsification handpiece 25 or into a disposable unit coupled to the handpiece (e.g., an anti-vacuum surge unit). In further examples, controller 60 and drive module 30 may be implemented as a single controller, where the power line for the ultrasound transducer may be connected to one of the outputs of the microcontroller.

The operation parameters and electrical current may be provided to ultrasound transducer 55 by drive module 30, using for example electrical wiring running within cable 33, subject to controller 60 enabling the current provisioning.

Referring now to FIG. 2B and FIG. 2C, showing schematic illustrations of needle 16 and lens capsule 18 of patient 19, and the activation-related components of the ultrasonic transducer.

In both FIG. 2B and FIG. 2C, impedance detection circuitry 62 applies a signal to the circuit comprising needle 16 and electrode 130. Impedance detection circuitry 62 may transmit a small-amplitude monitoring signal through cables 33 and 45, measure the voltage and determine the impedance. Alternatively, impedance detection circuitry 62 may apply voltage and measure the current. Impedance detection circuitry 62 may be adapted to detect impedance in the range of 20-150 Ohm, 50-120 Ohm, 20-120 Ohm, 50-150 Ohm, or the like.

If, as shown in FIG. 2B needle 16 is not in contact with lens capsule 18, the impedance therebetween as determined by impedance detection circuitry 62 and as shown in graph 52 is relatively low, for example about 70 Ohm.

However, if as shown in FIG. 2C needle 16 makes contact with lens capsule 18, the impedance therebetween as shown in graph 53 is high relative to graph 52, for example about 100 Ohm.

Impedance detection circuitry 62 may provide the measured impedance to controller 60, which determines, as described in FIG. 3 below, whether to toggle ultrasound transducer 55 ON/OFF.

In response to an indication that needle 16 has made contact with the eye lens, controller 60 may toggle ultrasound transducer 55 ON. In response to a signal indicating that needle 16 has lost contact with the eye lens, controller 60 may toggle ultrasound transducer 55 OFF. Upon ultrasound transducer 55 being toggled ON, ultrasound transducer 55 may vibrate needle 16 in one or more resonant vibration modes, to emulsify the cataract lens.

Referring now to FIG. 3, showing a flowchart of a method for operating an ultrasonic transducer, in accordance with some embodiments of the disclosure.

The method of FIG. 3 may be performed by controller 60, processor 38, or any other processor which may be configured to send a toggle signal to ultrasound transducer 55 or affect a power line connected thereto.

On step 300, an impedance reading may be obtained from impedance detection circuitry 62.

On step 304, it may be determined whether the impedance received on step 300 complies with a contact condition. For example, the condition may be whether the impedance is above a threshold, whether the impedance exceeds by at least a predetermined quantity or percentage relative to a previous impedance, whether the impedance derivative exceeds a threshold, or the like, as detailed above. Additionally or alternatively, contact may be determined by a trained AI engine, as detailed above. It may also be determined whether the ultrasonic transducer is off (i.e., whether the ultrasonic transducer has not been activated before, or has been stopped), meaning that the needle has just made contact with the lens capsule.

If the condition holds (“yes”), i.e., the transducer is off and the impedance indicates contact, then on step 308, the controller may toggle the transducer ON.

The method may then continue on step 300 with a subsequent impedance input.

However, if the condition does not hold (“no”), i.e., the transducer is already on, or the impedance does not comply with the condition, then on step 312 it is determined whether the condition does not hold and the transducer is on, meaning that needle 16 has just lost contact with lens capsule 18.

In this case (“yes”), on step 316 the transducer is toggled OFF.

If not, i.e., the condition holds or the transducer is off, but not both (since this combination has been checked on step 304), then either needle 16 is not in contact with lens capsule 18 and the transducer is OFF, or needle 16 is already in contact with lens capsule 18 and the transducer is ON. In these cases, no change is required in the operation status of the transducer, no toggling is performed, and execution may return to step 300 for obtaining a subsequent reading.

In some embodiments, processor 60 may also receive an indication of the pressure within aspiration channel 46a. If the pressure indicates that the channel is clogged, for example due to a block caused by an aspired piece of cataract, processor 60 may halt all vibrations until the block is released.

It is appreciated that the steps and modules disclosed above are in addition to the software, hardware, firmware or other modules required for operating the probe, displaying the phacoemulsification process, performing other calculations required for example for operating irrigation pump 24 or aspiration pump 26, or the like.

The method of FIG. 3 may be coded as a computer program product. The computer program product may include a computer readable storage medium (or media) having computer readable program instructions thereon for causing a processor to carry out aspects of the present invention.

The computer readable storage medium can be a tangible device that can retain and store instructions for use by an instruction execution device. The computer readable storage medium may be, for example, but is not limited to, an electronic storage device, a magnetic storage device, an optical storage device, an electromagnetic storage device, a semiconductor storage device, or any suitable combination of the foregoing. A non-exhaustive list of more specific examples of the computer readable storage medium includes the following: a portable computer diskette, a hard disk, a random access memory (RAM), a read-only memory (ROM), an erasable programmable read-only memory (EPROM or Flash memory), a static random access memory (SRAM), a portable compact disc read-only memory (CD-ROM), a digital versatile disk (DVD), a memory stick, a floppy disk, a mechanically encoded device such as punch-cards or raised structures in a groove having instructions recorded thereon, and any suitable combination of the foregoing. A computer readable storage medium, as used herein, is not to be construed as being transitory signals per se, such as radio waves or other freely propagating electromagnetic waves, electromagnetic waves propagating through a waveguide or other transmission media (e.g., light pulses passing through a fiber-optic cable), or electrical signals transmitted through a wire.

Computer readable program instructions described herein can be downloaded to respective computing/processing devices from a computer readable storage medium or to an external computer or external storage device via a network, for example, the Internet, a local area network, a wide area network and/or a wireless network. The network may comprise copper transmission cables, optical transmission fibers, wireless transmission, routers, firewalls, switches, gateway computers and/or edge servers. A network adapter card or network interface in each computing/processing device receives computer readable program instructions from the network and forwards the computer readable program instructions for storage in a computer readable storage medium within the respective computing/processing device.

Computer readable program instructions for carrying out operations of the present invention may be assembly instructions, instruction-set-architecture instructions, machine instructions, machine dependent instructions, microcode, firmware instructions, state-setting data, or either source code or object code written in any combination of one or more programming languages, programming languages such as Java, C, C++, Python, or others. The computer readable program instructions may execute entirely on the user's computer, partly on the user's computer, as a stand-alone software package, partly on the user's computer and partly on a remote computer or entirely on the remote computer or server. In the latter scenario, the remote computer may be connected to the user's computer through any type of network, including a local area network (LAN) or a wide area network (WAN), or the connection may be made to an external computer (for example, through the Internet using an Internet Service Provider). In some embodiments, electronic circuitry including, for example, programmable logic circuitry, field-programmable gate arrays (FPGA), or programmable logic arrays (PLA) may execute the computer readable program instructions by utilizing state information of the computer readable program instructions to personalize the electronic circuitry, in order to perform aspects of the present invention.

Aspects of the present invention are described herein with reference to flowchart illustrations and/or block diagrams of methods, apparatus (systems), and computer program products according to embodiments of the invention. It will be understood that each block of the flowchart illustrations and/or block diagrams, and combinations of blocks in the flowchart illustrations and/or block diagrams, can be implemented by computer readable program instructions.

These computer readable program instructions may be provided to a processor of a general-purpose computer, special purpose computer, or other programmable data processing apparatus to produce a machine, such that the instructions, which execute via the processor of the computer or other programmable data processing apparatus, create means for implementing the functions/acts specified in the flowchart and/or block diagram block or blocks. These computer readable program instructions may also be stored in a computer readable storage medium that can direct a computer, a programmable data processing apparatus, and/or other devices to function in a particular manner, such that the computer readable storage medium having instructions stored therein comprises an article of manufacture including instructions which implement aspects of the function/act specified in the flowchart and/or block diagram block or blocks.

The computer readable program instructions may also be loaded onto a computer, other programmable data processing apparatus, or other device to cause a series of operational steps to be performed on the computer, other programmable apparatus or other device to produce a computer implemented process, such that the instructions which execute on the computer, other programmable apparatus, or other device implement the functions/acts specified in the flowchart and/or block diagram block or blocks.

The flowchart and block diagrams in the Figures illustrate the architecture, functionality, and operation of possible implementations of systems, methods, and computer program products according to various embodiments of the present invention. In this regard, each block in the flowchart or block diagrams may represent a module, segment, or portion of instructions, which comprises one or more executable instructions for implementing the specified logical function(s). In some alternative implementations, the functions noted in the block may occur out of the order noted in the figures. For example, two blocks shown in succession may, in fact, be executed substantially concurrently, or the blocks may sometimes be executed in the reverse order, depending upon the functionality involved. It will also be noted that each block of the block diagrams and/or flowchart illustration, and combinations of blocks in the block diagrams and/or flowchart illustration, can be implemented by special purpose hardware-based systems that perform the specified functions or acts or carry out combinations of special purpose hardware and computer instructions.

### EXAMPLES

### Example 1

A phacoemulsification system, comprising: a phacoemulsification probe having a needle at its distal end, the needle configured to be inserted into an eye of a patient, the probe comprising an ultrasonic transducer; an impedance detection circuitry for determining an impedance between a tip of the needle and an electrode attached to the patient; and a processor, configured to repeatedly: obtain the impedance from the impedance detection circuitry; subject to the impedance complying with a condition and the ultrasonic transducer being inactive, toggling the ultrasonic transducer to transmit ultrasonic waves; and subject to the impedance not complying with a condition and the ultrasonic transducer being active, toggling the ultrasonic transducer to stop transmitting ultrasonic waves.

### Example 2

The phacoemulsification system according to example 1, wherein the processor is implemented as a microcontroller.

### Example 3

The phacoemulsification system according to example 1, wherein the condition is that the impedance exceeds a threshold.

### Example 4

The phacoemulsification system according to example 1, wherein the condition is that the impedance is higher in at least a predetermined quantity or percent than a previous impedance.

### Example 5

The phacoemulsification system according to example 1, wherein the condition is that a derivative of the impedance exceeds a threshold.

### Example 6

The phacoemulsification system according to example 1, wherein whether the condition holds is determined by a trained artificial intelligence engine.

### Example 7

The phacoemulsification system according to example 1, wherein the impedance is detected by applying current to a circuit comprising the tip of the needle and the electrode, measuring voltage in the circuit, and dividing the voltage by the current.

### Example 8

The phacoemulsification system according to example 8, wherein the current is between about 5 μamp and 100 μamp, and at a frequency of about 50 KHz.

### Example 9

The phacoemulsification system according to example 1, wherein the impedance is detected by applying voltage to a circuit comprising the tip of the needle and the electrode, measuring a current within the circuit, and dividing the voltage by the current.

### Example 10

The phacoemulsification system according to example 1, wherein the impedance threshold is between about 20 Ohm and 150 Ohm.

### Example 11

The phacoemulsification system according to example 1, further comprising: an irrigation channel for irrigating the eye with irrigation fluid; an irrigation sensor, which is coupled with the irrigation channel and is configured to measure a parameter indicative of a pressure of the irrigation fluid; and an irrigation pump configured to flow the irrigation fluid to the irrigation channel.

### Example 12

The phacoemulsification system of claim 1, further comprising: an aspiration channel for evacuating material from the eye; an aspiration sensor, coupled with the aspiration channel and configured to measure a value indicative of a pressure in the aspiration channel; and an aspiration pump configured to evacuate the material from the aspiration channel.

### Example 13

A method for applying phacoemulsification to an eye of a patient, comprising: obtaining an impedance between an electrode attached to the patient and a tip of a needle of a phacoemulsification probe, when the needle is inserted into the eye of the patient; subject to the impedance complying with a condition and an ultrasonic transducer comprised in the phacoemulsification probe being inactive, toggling the ultrasonic transducer to transmit ultrasonic waves; and subject to the impedance not complying with a condition and the ultrasonic transducer being active, toggling the ultrasonic transducer to stop transmitting ultrasonic waves.

### Example 14

A computer program product comprising a non-transitory computer readable medium retaining program instructions, which instructions when read by a processor, cause the processor to perform: obtaining an impedance between an electrode attached to a patient and a tip of a needle of a phacoemulsification probe, when the needle is inserted into an eye of the patient; subject to the impedance complying with a condition and an ultrasonic transducer comprised in the phacoemulsification probe being inactive, toggling the ultrasonic transducer to transmit ultrasonic waves; and subject to the impedance not complying with a condition and the ultrasonic transducer being active, toggling the ultrasonic transducer to stop transmitting ultrasonic waves.

Although the examples described herein mainly address cardiac diagnostic applications, the methods and systems described herein can also be used in other medical applications.

It will be appreciated that the examples described above are cited by way of example, and that the present disclosure is not limited to what has been particularly shown and described hereinabove. Rather, the scope of the present disclosure includes both combinations and subcombinations of the various features described hereinabove, as well as variations and modifications thereof which would occur to persons skilled in the art upon reading the foregoing description and which are not disclosed in the prior art.

1. A phacoemulsification system, comprising:
a phacoemulsification probe having a needle at its distal end, the needle configured to be inserted into an eye of a patient, the probe comprising an ultrasonic transducer;
an impedance detection circuitry for determining an impedance between a tip of the needle and an electrode attached to the patient; and
a processor, configured to repeatedly:
obtain the impedance from the impedance detection circuitry;
subject to the impedance complying with a condition and the ultrasonic transducer being inactive, toggling the ultrasonic transducer to transmit ultrasonic waves; and
subject to the impedance not complying with a condition and the ultrasonic transducer being active, toggling the ultrasonic transducer to stop transmitting ultrasonic waves.

2. The phacoemulsification system of claim 1, wherein the processor is implemented as a microcontroller.

3. The phacoemulsification system of claim 1, wherein the condition is that the impedance exceeds a threshold.

4. The phacoemulsification system of claim 1, wherein the condition is that the impedance is higher in at least a predetermined quantity or percent than a previous impedance.

5. The phacoemulsification system of claim 1, wherein the condition is that a derivative of the impedance exceeds a threshold.

6. The phacoemulsification system of claim 1, wherein whether the condition holds is determined by a trained artificial intelligence engine.

7. The phacoemulsification system of claim 1, wherein the impedance is detected by applying current to a circuit comprising the tip of the needle and the electrode, measuring voltage in the circuit, and dividing the voltage by the current.

8. The phacoemulsification system of claim 7, wherein the current is between about 5 μamp and 100 μamp, and at a frequency of about 50 KHz.

9. The phacoemulsification system of claim 1, wherein the impedance is detected by applying voltage to a circuit comprising the tip of the needle and the electrode, measuring a current within the circuit, and dividing the voltage by the current.

10. The phacoemulsification system of claim 1, wherein the impedance threshold is between about 20 Ohm and 150 Ohm.

11. The phacoemulsification system of claim 1, further comprising:
an irrigation channel for irrigating the eye with irrigation fluid;
an irrigation sensor, which is coupled with the irrigation channel and is configured to measure a parameter indicative of a pressure of the irrigation fluid; and
an irrigation pump configured to flow the irrigation fluid to the irrigation channel.

12. The phacoemulsification system of claim 1, further comprising:
an aspiration channel for evacuating material from the eye;
an aspiration sensor, coupled with the aspiration channel and configured to measure a value indicative of a pressure in the aspiration channel; and
an aspiration pump configured to evacuate the material from the aspiration channel.

13. A method for applying phacoemulsification to an eye of a patient, comprising:
obtaining an impedance between an electrode attached to the patient and a tip of a needle of a phacoemulsification probe, when the needle is inserted into the eye of the patient;
subject to the impedance complying with a condition and an ultrasonic transducer comprised in the phacoemulsification probe being inactive, toggling the ultrasonic transducer to transmit ultrasonic waves; and
subject to the impedance not complying with a condition and the ultrasonic transducer being active, toggling the ultrasonic transducer to stop transmitting ultrasonic waves.

14. A computer program product comprising a non-transitory computer readable medium retaining program instructions, which instructions when read by a processor, cause the processor to perform:
obtaining an impedance between an electrode attached to a patient and a tip of a needle of a phacoemulsification probe, when the needle is inserted into an eye of the patient;
subject to the impedance complying with a condition and an ultrasonic transducer comprised in the phacoemulsification probe being inactive, toggling the ultrasonic transducer to transmit ultrasonic waves; and
subject to the impedance not complying with a condition and the ultrasonic transducer being active, toggling the ultrasonic transducer to stop transmitting ultrasonic waves.
